(19) United States
(12) Patent Application Publication
(10) Pub. No.: US 2009/0024106 A1
(43) Pub. Date: Jan. 22, 2009

(54) Method and apparatus for maintaining access

(76) Inventor: Edward J. Morris, Bloomington, IN (US)

Correspondence Address: BAKER & DANIELS LLP, 300 NORTH MERIDIAN STREET, SUITE 2700, INDIANAPOLIS, IN, 46204, US

(21) Appl. No.: 11/879,426

(22) Filed: Jul. 17, 2007

(51) Int. Cl. A61M 25/02

(52) U.S. Cl. 604/508; 604/175; 606/108

(57) ABSTRACT

An apparatus and a method for sealing a puncture in a tubular tissue structure or the wall of a body cavity are provided.

The present disclosure relates to an apparatus and a method for maintaining a passageway in tissue or a body cavity. More particularly, the present disclosure is directed to maintaining a passageway with submucosal tissue or another extracellular matrix-derived tissue capable of remodeling endogenous connective tissue or with a synthetic bioabsorbable material.

### BACKGROUND AND SUMMARY

The control of bleeding during and after surgery as well as prevention of infection during and after surgery is important to the success of the procedure. A number of procedures involve “tunnelling” of catheters or other tubular structures beneath the skin over distances. Typically, the insertion of a catheter creates a puncture through tissue and a wall of a cavity such as a vessel wall until the catheter reaches a cavity or location to which access is desired. Such tunnelling exposes a relatively large amount of tissue to the catheter compared to more direct access.

Many tunneling applications involve the replacement of a first catheter with a subsequent catheter in the same location for the same or different purpose. Accordingly, tunnelling procedures create relatively large tissue exposure to the catheter and relatively large sites for potential infection.

Accordingly, there is a need for surgical techniques suitable for reducing infection sites and infection susceptibility in tunneling procedures.

In one embodiment, a device for maintaining access to a bodily cavity is provided. The device comprises an elongated element having a tissue wall contact exterior portion and having a length adapted to be inserted into a puncture site so that the length forms intravascular, intermediate, and extracorporeal portions, and a bioabsorbable member releasably attached to the tissue wall contact exterior portion of the elongated element, the bioabsorbable member having intravascular, intermediate, and extracorporeal portions.

In another embodiment, a device for maintaining an access site to a bodily cavity is provided. The device comprises an elongated element having a tissue wall contact exterior portion and a bioabsorbable member releasably attached to the tissue wall contact exterior portion of the elongated element, the bioabsorbable member having a length to extend from within the cavity to the skin of a body.

In an alternate embodiment, a method of maintaining an access point in tissue is provided. The method comprises the step of providing a bioabsorbable member at the access point such that the bioabsorbable member extends from outside the body to within a bodily cavity.

In another embodiment, a method of maintaining an access point in tissue is provided. The method comprises the steps of providing a first elongated element having a bioabsorbable member disposed on the exterior thereof, the first elongated element being configured to be introduced into a body with the bioabsorbable member disposed thereon, removing the first elongated element, and placing a second elongated element within the bioabsorbable member.

### BRIEF DESCRIPTION OF THE DRAWINGS

FIG. 1 illustrates an introducer element including a sheet for use in obtaining and maintaining access to a tubular tissue structure or a body cavity;

FIG. 2 illustrates a tether configuration between the introducer element and the sheet for use in placing the combination introducer element and sheet in contact with a tubular tissue structure or a body cavity;

FIG. 3 illustrates the introducer and sheet of FIG. 1 having gained access to a tubular tissue structure or a body cavity and being positioned at least partially therein; and

FIG. 4 illustrates the tubular tissue structure or body cavity of FIG. 3 with the introducer removed and the sheet retained therein.

### DETAILED DESCRIPTION

The present disclosure is related to an apparatus and a method for maintaining access to a tubular tissue structure, such as a blood vessel, or in the wall of a body cavity, with submucosal tissue, another extracellular matrix-derived tissue, or a synthetic bioabsorbable material capable of supporting the growth of endogenous connective tissue in vivo resulting in remodeling of endogenous connective tissue at the puncture site, all of these materials and the like are referred to generally as bioabsorbable materials. The apparatus and method of the present disclosure can be used to maintain and an access point to a tubular tissue structure, such as a blood vessel, or in the wall of another body cavity. The apparatus further provides a surface along an access passageway such that raw tissue is not appreciably exposed. Whereas the device and method of using will be described herein with a Broviac/Hickman catheter for use in subclavian dialysis, the device may be used on many other medical devices in many other medical procedures. While not exhaustive, such other medical devices may include central lines, port-a-caths, ventriculo-peritoneal shunts, ventriculo-atrial shuts, Quinton catheters, pumps such as Baclofen pumps or opiate delivering pumps, and Groshong catheters. Additionally, any applications where tubing is tunneled within the anatomy may find useful application of the present disclosure.

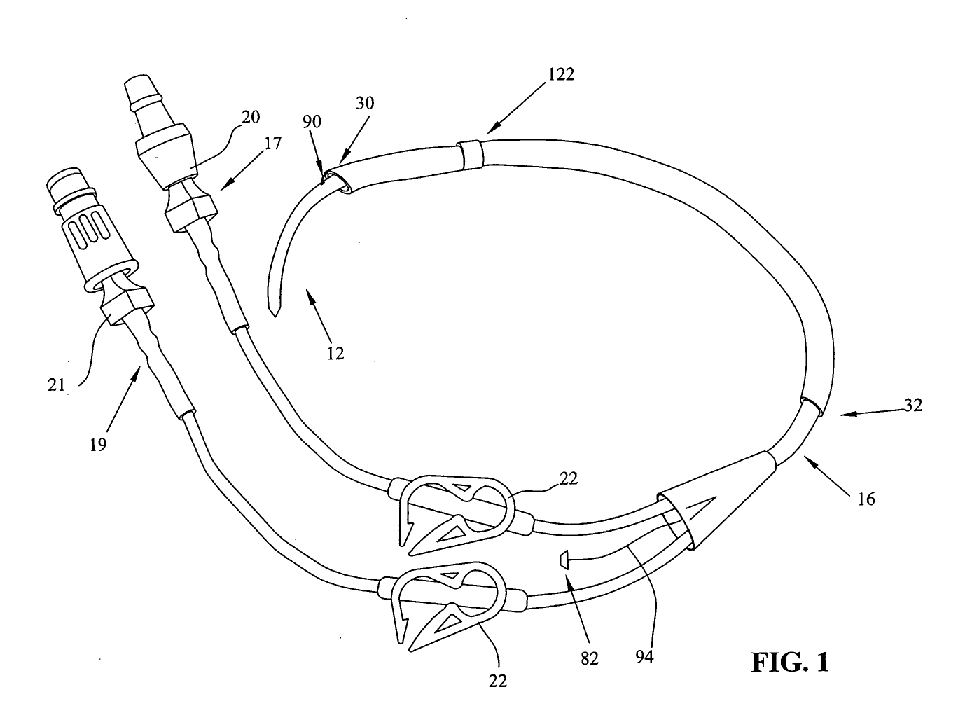

Referring now to the drawings, FIG. 1 illustrates a catheter 10 for use with an introducer (not shown) adapted for catheterization, exemplary of the type of catheter element that may be used in accordance with the present disclosure. Although catheter 10 is illustrated in FIG. 1, it is understood that the present disclosure is applicable to any type of catheter or introducer element used to provide access to the lumen of a tubular tissue structure, such as a blood vessel, or to another body cavity. For example, the present disclosure is applicable to a catheter element, a needle, a cannula, a guide wire, an introducer element adapted for dialysis, a trocar, or any other introducer element used to access the lumen of a tubular tissue structure or a body cavity.

Catheter 10 as depicted in FIG. 1 is used when performing catheterization procedures in coronary and peripheral veins for dialysis. As previously noted, other implementations obtaining access to arteries and other bodily cavities are also envisioned. Typically, catheter 10 is introduced into the vascular system by first penetrating the skin, underlying muscle tissue, and the blood vessel with a needle (not shown), and a guide wire (not shown) is inserted through the lumen of the needle and enters the blood vessel. Subsequently, the needle is stripped off the guide wire and catheter 10 is fed over the guide wire and tunneled through the skin and through the vessel wall to enter the vessel. The guide wire can then be removed and catheter 10 is advanced through the vascular system until the working end of catheter 10 is positioned at a predetermined location. Alternatively, the guide wire may be left in place throughout the procedure. Furthermore any embodiment of catheter 10 described below is applicable to any other introducer element for use in accessing the lumen of a tubular tissue structure or a body cavity.

Catheter 10 is designed and intended to remain within the patient for extended periods of time. Catheter 10 illustrated in FIG. 1 is an exemplary embodiment and has user distal end 12 for insertion into a blood vessel and user proximal end 14. Catheter 10 includes sheath 16, a pair of supply/return tubes 17, 19, ratchet clamps 22 and caps 20, 21 disposed axially over tubes 17, 19. Lumen 104 is defined in sheath 16. Although not part of a standard catheter, catheter 10 depicted in FIG. 1 further comprises sheet 18 of submucosal tissue or another extracellular matrix-derived tissue or a synthetic bioabsorbable material extending axially over a portion of sheath 16. Sheet 18 includes a type of tactile stop, sleeve cuff 122.

The submucosal tissue, another extracellular matrix-derived tissue, or a synthetic bioabsorbable material can be in the form of a ribbon with unjoined edges, a cylindrically-shaped tube with joined edges, a roll wrapped multiple times around catheter 10, or in any other suitable form.

Exemplary of tissues that can be used to make sheet 18 are submucosal tissues or any other bioabsorbable materials (e.g., an extracellular matrix-derived tissue of a warm-blooded vertebrate). Submucosal tissue can comprise submucosal tissue selected from the group consisting of intestinal submucosa, stomach submucosa, urinary bladder submucosa, and any other submucosal tissue that is acellular and can be used to remodel endogenous tissue. The submucosal tissue can comprise the tunica submucosa delaminated from both the tunica muscularis and at least the luminal portion of the tunica mucosa of a warm-blooded vertebrate.

It is known that compositions comprising the tunica submucosa delaminated from both the tunica muscularis and at least the luminal portion of the tunica mucosa of the submucosal tissue of warm-blooded vertebrates can be used as tissue graft materials (see, for example, U.S. Pat. Nos. 4,902,508 and 5,281,422 incorporated herein by reference). Such submucosal tissue preparations are characterized by excellent mechanical properties, including high compliance, high tensile strength, a high burst pressure point, and tear-resistance. Thus, sheets 18 prepared from submucosal tissue are tear-resistant preventing occlusive material from being disposed into the blood vessel.

Submucosal tissue sheets provide resistance to infection, stability, and lack of immunogenicity. Intestinal submucosal tissue, fully described in the aforesaid patents, has high infection resistance. In fact, most of the studies done with intestinal submucosa grafts to date have involved non-sterile grafts, and no infection problems have been encountered. Of course, appropriate sterilization techniques can be used to treat submucosal tissue. Furthermore, this tissue is not recognized by the host's immune system as “foreign” and is not rejected. It has been found that xenogeneic intestinal submucosa is not rejected following implantation as vascular grafts, ligaments, and tendons because of its composition (i.e., submucosal tissue is apparently similar among species). It has also been found that submucosal tissue has a long shelf-life and remains in good condition for at least two months at room temperature without any resultant loss in performance.

Submucosa-derived matrices are collagen based biodegradable matrices comprising highly conserved collagens, glycoproteins, proteoglycans, and glycosaminoglycans in their natural configuration and natural concentration. Such submucosal tissue used as a sheet 18 on a catheter element serves as a matrix for the regrowth of endogenous connective tissues (i.e., biological remodeling, bonding, begin to occur upon insertion of the introducer element with submucosal tissue sheet 18 into the tissue). Submucosal tissue sheet 18 serves as a rapidly vascularized matrix for support and growth of new endogenous connective tissue. Thus, submucosal tissue has been found to be trophic for host tissues with which it is attached or otherwise associated in its implanted environment. In multiple experiments submucosal tissue has been found to be remodeled (resorbed and replaced with autogenous differentiated tissue) to assume the characterizing features of the tissue(s) with which it is associated at the site of implantation or insertion. Additionally, the boundaries between the submucosal tissue and endogenous tissue are not discernible after remodeling. Thus, submucosal tissue may be used as a connective tissue substitute, particularly to remodel tissue along the path of a catheter.

Small intestinal tissue is a suitable source of submucosal tissue for use in this disclosure. Submucosal tissue can be obtained from various sources, for example, intestinal tissue can be harvested from animals raised for meat production, including, pigs, cattle and sheep or other warm-blooded vertebrates. Small intestinal submucosal tissue is a plentiful by-product of commercial meat production operations and is, thus, a low cost material.

Suitable intestinal submucosal tissue typically comprises the tunica submucosa delaminated from both the tunica muscularis and at least the luminal portion of the tunica mucosa. In one embodiment the intestinal submucosal tissue comprises the tunica submucosa and basilar portions of the tunic a mucosa including the lamina muscularis mucosa and the stratum compactum which layers are known to vary in thickness and in definition dependent on the source vertebrate species.

The preparation of submucosal tissue is described in U.S. Pat. No. 4,902,508, the disclosure of which is expressly incorporated herein by reference. A segment of vertebrate intestine, for example, harvested from porcine, ovine or bovine species, but not excluding other species, is subjected to abrasion using a longitudinal wiping motion to remove the outer layers, comprising smooth muscle tissues, and the innermost layer, i.e., the luminal portion of the tunica mucosa. The submucosal tissue is rinsed with saline and is optionally sterilized.

The submucosal tissue for use as sheet 18 on catheter element 10 can be sterilized using conventional sterilization techniques including glutaraldehyde tanning, formaldehyde tanning at acidic pH, propylene oxide or ethylene oxide treatment, gas plasma sterilization, gamma radiation, electron beam, peracetic acid sterilization. Sterilization techniques which do not adversely affect the mechanical strength, structure, and biotropic properties of the submucosal tissue are suitable. For instance, strong gamma radiation may cause loss of strength of the sheets of submucosal tissue. Sterilization techniques include exposing the submucosal tissue sheet to peracetic acid, 1-4 Mrads gamma irradiation (alternatively 1-2.5 Mrads of gamma irradiation), ethylene oxide treatment or gas plasma sterilization.

Typically, the submucosal tissue is subjected to two or more sterilization processes. After the submucosal tissue is sterilized, for example, by chemical treatment, the tissue can be wrapped in a plastic or foil wrap, for example, as packaging for the preparation, and sterilized again using electron beam or gamma irradiation sterilization techniques. Alternatively, the catheter element can be assembled with submucosal tissue sheet 18 on the catheter element and the complete assembly can be packaged and sterilized a second time.

The submucosal tissue can be stored in a hydrated or dehydrated state. Lyophilized or air dried submucosa tissue can be rehydrated and used without significant loss of its biotropic and mechanical properties. The submucosal tissue can be rehydrated before use or, alternatively, is rehydrated during use upon insertion through the skin and into the tubular tissue structure, such as a blood vessel, or a body cavity.

The submucosal tissue can be conditioned, as described in U.S. Pat. No. 5,275,826 (the disclosure of which is expressly incorporated herein by reference) to alter the viscoelastic properties of the submucosal tissue. In accordance with one embodiment submucosa tissue delaminated from the tunica muscularis and luminal portion of the tunica mucosa is conditioned to have a strain of no more than 20%. The submucosal tissue is conditioned by stretching, chemically treating, enzymatically treating or exposing the tissue to other environmental factors. In one embodiment the submucosal tissue is conditioned by stretching in a longitudinal or lateral direction so that the submucosal tissue has a strain of no more than 20%.

When a segment of intestine is first harvested and delaminated as described above, it will be a tubular segment having an intermediate portion and opposite end portions. To form submucosal tissue sheets 18, sheets of delaminated submucosal tissue can be cut from this tubular segment of intestine to form squares or rectangles of the desired dimensions. The edges of the squares or rectangles can be overlapped and can be joined to form a tubular structure or the edges can be left unjoined. In embodiments where the edges are left unjoined, sheet 18 can be held in place on sheath 16, for example. Thus, sheet 18 can be in the form of a ribbon with unjoined edges, a tubular structure with overlapped, joined edges, a roll of tissue wrapped around sheath 16 multiple times, a disk, as described above, or in any other form suitable for use in accordance with the present disclosure. Such embodiments of sheet 18 are applicable to submucosal tissue or to other extracellular matrix-derived tissues, or to synthetic bioabsorbable materials and to use with any type of introducer element.

In one embodiment, the edges of the prepared squares or rectangles can be overlapped and joined to form cylinder-shaped submucosal tissue sheet 18 with the desired diameter. The edges can be joined and a cylinder-shaped sheet formed by applying pressure to sheet 18 including the overlapped portions by compressing the submucosal tissue between two surfaces. The two surfaces can be formed from a variety of materials and in any cylindrical shape depending on the desired form and specification of sheet 18. Typically, the two surfaces used for compression are formed as a cylinder and a complementary nonplanar curved plate. Each of these surfaces can optionally be heated or perforated. In certain embodiments at least one of the two surfaces is water permeable. The term water permeable surface as used herein includes surfaces that are water absorbent, microporous or macroporous. Macroporous materials include perforated plates or meshes made of plastic, metal, ceramics or wood.

The submucosal tissue is compressed in accordance with one embodiment by placing sheet 18 including the overlapped portions of the sheets of submucosal tissue on a first surface (i.e., inserting a cylinder of the desired dimensions in a cylinder of submucosal tissue) and placing a second surface on top of the exposed submucosal surface. A force is then applied to bias the two surfaces (i.e., the plates) towards one another, compressing the submucosal tissue between the two surfaces. The biasing force can be generated by any number of methods known to those skilled in the art including the application of a weight on the top plate, and the use of a hydraulic press or the application of atmospheric pressure on the two surfaces.

In one embodiment the strips of submucosal tissue are subjected to conditions permitting dehydration of the submucosal tissue concurrent with the compression of the tissue. The term “conditions permitting dehydration of the submucosal tissue” is defined to include any mechanical or environmental condition which promotes or induces the removal of water from the submucosal tissue at least at the points of overlap. To promote dehydration of the compressed submucosal tissue, at least one of the two surfaces compressing the tissue can be water permeable. Dehydration of the tissue can optionally be further enhanced by applying blotting material, heating the tissue or blowing air across the exterior of the two compressing surfaces.

The submucosal tissue is typically compressed for 12-48 hours at room temperature, although heat may also be applied. For example, a warming blanket can be applied to the exterior of the compressing surfaces to raise the temperature of the compressed tissue up to about 50° C. to about 400° C. The overlapped portions are usually compressed for a length of time determined by the degree of dehydration of the tissue. The use of heat increases the rate of dehydration and thus decreases the amount of time the submucosal tissue is required to be compressed. Sufficient dehydration of the tissue is indicated by an increase in impedance of electrical current flowing through the tissue. When impedance has increased by 100-200 ohms, the tissue is sufficiently dehydrated and the pressure can be released.

A vacuum can optionally be applied to submucosal tissue during the compression procedure. The applied vacuum enhances the dehydration of the tissue and may assist the compression of the tissue. Alternatively, the application of a vacuum can provide the sole compressing force for compressing the submucosal tissue including the overlapped edges. For example, the submucosal tissue can be placed between two surfaces, optionally one of which is water permeable. The apparatus is covered with blotting material, to soak up water, and a breather blanket to permit air flow. The apparatus is then placed in a vacuum chamber and a vacuum is applied, generally ranging from 14-70 inches of Hg (7-35 psi). In some embodiments, a vacuum is applied at approximately 51 inches of Hg (25 psi). Optionally a heating blanket can be placed on top of the chamber to heat the submucosal tissue during the compression of the tissue. Chambers suitable for use in this embodiment are known to those skilled in the art and include any device that is equipped with a vacuum port. The resulting drop in atmospheric pressure coacts with the two surfaces to compress the submucosal tissue and simultaneously dehydrate the submucosal tissue. The compressed submucosal tissue can be removed from the two surfaces as a cylinder. The construct can be further manipulated (i.e., tethers can be attached) as described above.

In alternate embodiments, the overlapped portions of the submucosal tissue sheet or extracellular matrix-derived material or synthetic material can be attached to each other by suturing with resorbable thread or by any other method of bonding the overlapped edges known to a person skilled in the art. Such methods of attaching the overlapped edges of the sheet to each other can be used with or without compression to form, for example, a cylindrically-shaped tube, a roll, or a disk. Sheet 18 can also be formed from multiple layers of submucosal tissue attached to each other by compression as described above. The diameter of sheet 18 can vary depending on the desired specifications of the sheet. For example, the diameter of sheet 18 can be from about 3 to about 12 french when sheet 18 is used on an introducer element adapted for catheterization but any diameter can be used depending on the diameter of the introducer element.

Methods of preparing other extracellular matrix-derived tissues are known to those skilled in the art and may be similar to those described above for submucosal tissue. For example, see WO 01/45765 and U.S. Pat. No. 5,163,955, incorporated herein by reference. Extracellular matrix-derived tissues include such tissue preparations as liver basement membrane, pericardial tissue preparations, sheet-like collagen preparations, denatured collagen, gelfoam, and the like.

In another illustrative embodiment, synthetic materials can be used to form sheet 18. Synthetic materials that can be used include biodegradable polymers such as polylactic acid (PLA), polyglycolic acid (PGA), poly(lactic acid-glycolic acid) copolymer (PLGA), poly-ε-caprolactone (PCL), poly(glycolic acid-caprolactone) copolymer (PGCL), polyanhydride, polyorthoester, and copolymers and mixtures thereof. Additional suitable materials include: collagen, gelatin, thrombin, synthetic protein based materials including alginate polysaccharides, polysaccaride films, lipids, sorbitol, glycerol, polypetides, and any pro-coagulant material. The biodegradable polymers and other materials can be, for example, in the form of a film, a sheet, a tube, a disk, a roll, or a ribbon. Illustratively, the materials can be woven and can be expandable or nonexpandable. The materials should be bioabsorbable, nonimmunogenic, and tear-resistant. Mixtures of the submucosal tissues, extracellular matrix-derived tissues, synthetic materials, and other materials can also be used.

In yet other illustrative embodiments, any of the extracellular matrix-derived tissues, the submucosal tissue preparations, or the synthetic materials described above, can be impregnated with biological response modifiers such as glycoproteins, glycosaminoglycans, chondroitin compounds, laminin, poly-n-acetyl glucosamine, chitosan, chondroitin, zeolite, potato starch, tranexamic acid, aminocaproic acid, desmopressin acetate, crushed collagen, gelfoam, clotting agents or clot protectors, such as thrombin, fibrin, fibrinogen, anti-fibrinolytics, factors VII, VIII, XIII, and the like, procoagulants, barriers, tissue factor, or blood factors, growth factors, and the like, or combinations of these biological response modifiers. These biological response modifiers can be placed at any effective location on the sheet 18, such as at distal end 30 of the sheet, at proximal end 32 of sheet 18, or under cuff 122.

In another illustrative embodiment, a radiopaque material can be incorporated into any of the extracellular matrix-derived tissues, the submucosal tissue preparations, or the synthetic materials described above used to make sheet 18. A radiopaque material can also be incorporated into any tether described above. Incorporation of a radiopaque material makes sheet 18 and/or tether visible under a fluoroscope, for example. In such an embodiment, the placement of sheet 18 and or the tether can be confirmed by the physician. The access site location can also be determined in the event that the patient undergoes another surgical procedure at a later time.

In various illustrative embodiments, the radiopaque material can be a barium salt such as barium sulfate, barium fluoride, or barium polyacrylate, bismuth oxychloride, bismuth trioxide, titanium dioxide, zirconium oxide, zirconium dioxide, chromium oxide, zinc oxide, or other metal oxides, bismuth glass, or mixtures of any of these radiopaque materials, or any other radiopaque materials known in the art. The radiopaque material(s) can be incorporated into the extracellular matrix-derived tissues, the submucosal tissue preparations, or the synthetic materials by procedures known to those skilled in the art such as dipping, coating, laminating, or encapsulating. In another illustrative embodiment, radiopaque marks, such as stripes and/or dots, can be placed strategically to locate distal end 30 of sheet 18, proximal end 32 of sheet 18, or sleeve cuff 122, for example.

In another illustrative embodiment, radiopaque marks (e.g., bands, dots, dashes, and the like) can be used to mark sheath 16 to aid the physician in visualizing sheath 16 and sheath 16 to sheet 18 placement. In another embodiment, or in addition to marking sheath 16, radiopaque marks can be placed on catheter 10 or on an access needle to determine the depth of the vessel and to indicate the proper placement of sheet 18 at the vessel. In other embodiments, radiopaque marks can be used to mark any other component of the device described herein. Any of the radiopaque materials described herein or any other radiopaque materials known in the art can be used to mark one or more components of the device described herein.

In other illustrative aspects, mannitol or other pastes known in the art, or a biocompatible liquid or solid lubricant can be added to distal end 30 of sheet 18. Pastes or biocompatible liquid or solid lubricants, for example, will provide a means of preventing distal end 30 of sheet 18 from rolling up upon insertion of the introducer with sheet 18 into the patient by serving as a transition between sheath 16 and sheet 18 during insertion of the device into the patient. Mannitol and similar pastes, for example, will also be safely and rapidly dissolved during/after insertion of the introducer with sheet 18 into the patient. The pastes and biocompatible lubricants should be capable of being sterilized by conventional techniques (e.g., autoclaving, filtering, irradiation) used for sterilizing pharmaceuticals and medical devices, and can be applied in the form of a liquid or gel, for example. Illustrative biocompatible lubricants include hyaluronic acid, dextran sulfate, dextran, succinylated noncrosslinked collagen, methylated non-cross-linked collagen, glycogen, glycerol, dextrose, maltose, and triglycerides.

In the embodiment of the disclosure depicted in FIG. 1, sheet 18 of submucosal tissue or another extracellular matrix-derived tissue or a synthetic bioabsorbable material extends axially over a portion of sheath 16. FIG. 5 depicts sheath 16, tubes 17, 19, and the sheet 18 in a disassembled cross-sectional form, and assembled to construct an catheter 10. Sheet 18 has user distal end 30 which is inserted into a tubular tissue structure, such as a blood vessel, and user proximal end 32 which remains outside of the punctured vessel wall. The proximal end 32 of the sheet 18 may extend axially over a portion of the sheath 16 as depicted in FIG. 1 or may extend to the proximal end of sheath 16.

In embodiments where user proximal end 32 of sheet 18 does not extend to the proximal end of sheath 16, user proximal end 32 of sheet 18 may be held in place, for example, by a string attached to user proximal end 32 of sheet 18 and sheath 16. As a result, sheet 18 is prevented from being pushed down catheter 10 when the user inserts catheter 10 through, for example, a vessel wall with his hand in contact with sheet 18. The string may be cut to permit user proximal end 32 of sheet 18 to move if desired. In other embodiments, user proximal end 32 of sheet 18 or other parts of sheet 18 may be held in place by metal or plastic clamps, O-rings, or the like, which may be removed from the end of sheet 18 when necessary. Alternatively, as shown in FIG. 1, sheet 18 may extend axially over only a portion of sheath 16 so that proximal end 32 of sheet 18 is distal to the points at which the hand of user contacts sheath 16 and does not come in contact with the hand of the user when catheter 10 is being inserted through the vessel wall. Sheet 18 can be of any length as long as sheet 18 is of sufficient length to line and abut the tissue proximate catheter 10 as it extends from the external puncture site to within the vein, (or other chosen body cavity).

Although not depicted in FIG. 1, in one embodiment user distal end 30 of sheet 18 is tapered from user distal end 30 towards user proximal end 32 to prevent sheet 18 from rolling up sheath 16 upon insertion into the blood vessel when sheet 18 is positioned, as shown in FIG. 3 during insertion into the blood vessel. Although, sheet 18 includes tether 90 as depicted in FIG. 1 and described below, any configuration of user distal end 30 of sheet 18 can be used which prevents sheet 18 from rolling up catheter 10 upon insertion into the blood vessel.

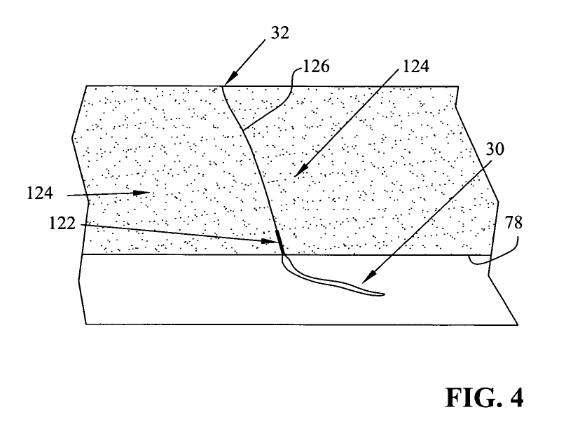

As shown in FIGS. 1-4, an embodiment for preventing sheet 18 from rolling up sheath 16 upon insertion into a tubular tissue structure is shown. Retaining wire 94 is attached to cap 87. Cap 87 can be screwed onto, snapped onto, or otherwise attached to, sheath 16 to hold retaining wire 94 in place in lumen 104.

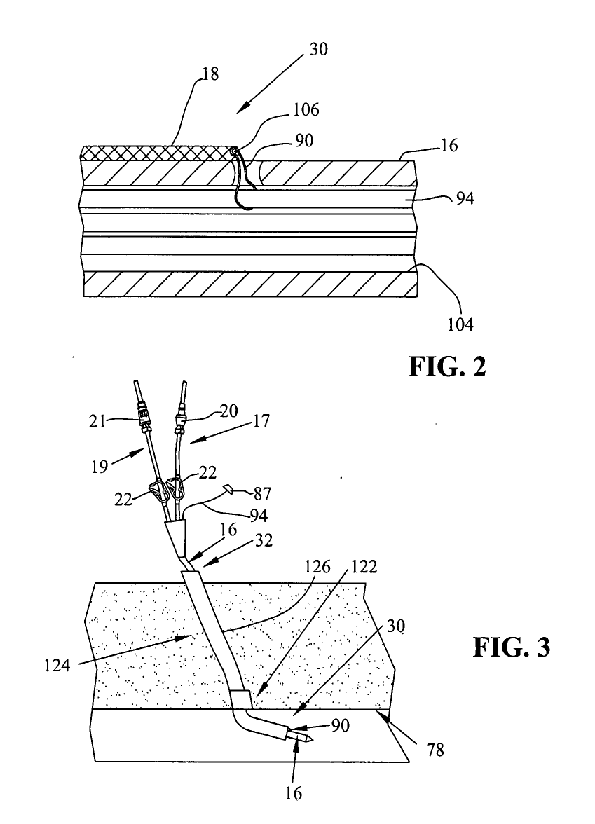

When retaining wire 94 is inserted into lumen 104, prior to arriving at the surgical application, retaining wire 94 is threaded through tether 90 in the form of a loop attached to distal end 30 of sheet 18 at attachment point 106 (see FIG. 2). Tether 90 can be attached to sheet 18, for example, by tying tether 90 to form a knot. Tether 90 extends radially inwards into lumen 104 through access port 92 defined in the wall of sheath 16.

Accordingly, tether 90, anchored by retaining wire 94, prevents sheet 18 from rolling up sheath 16 upon insertion into tissue. After insertion of catheter 10 with the sheet 18 through tissue and the wall of the tubular tissue structure, retaining wire 94 can be removed from lumen 104 by releasing cap 87 from catheter 10 and by pulling retaining wire 94, attached to cap 87, out of lumen 104. Thus, tether 90 is no longer anchored by retaining wire 94. Catheter 10 can then move relative to sheet 18. While movement of catheter 10 is minimized, any incidental movement is not necessarily translated to sheet 18. Accordingly, attachment of remodeling sheet 18 to surrounding tissue is more likely.

Various additional parts, tether locations, and anti-roll up features are described in U.S. patent application Ser. No. 11/546,066 titled Method and apparatus for sealing access, the disclosure of which is expressly incorporated herein by reference. Such application describes introducers with many similar parts to catheter 10, it should be appreciated that the various parts of the introducers may be substituted for similar parts of catheter 10.

The present disclosure is also directed to a method of maintaining an access site in tissue. The method comprises the step of inserting submucosal tissue or another intact extracellular matrix-derived tissue of a warm-blooded vertebrate or a synthetic bioabsorbable material into tissue. In accordance with the disclosure, “intact extracellular matrix-derived tissue” means an extracellular matrix-derived tissue at least a portion of which is in its native three-dimensional configuration. The tissue can be in the form of, for example, a ribbon, a cylindrically-shaped tube, a disk, or a roll and can be inserted into the puncture site in the form of sheet 18 on any type of introducer element used to provide access to the lumen of a tubular tissue structure or to access a body cavity.

In one embodiment the method comprises the step of inserting a catheter element into the puncture site. An exemplary embodiment is depicted in FIG. 3 and catheter 10 has sheet 18 comprising submucosal tissue or another extracellular matrix-derived tissue of a warm-blooded vertebrate or a synthetic bioabsorbable material and sheet 18 has user distal end 30 and user proximal end 32. User proximal end 32 of the sheet 18 remains outside of the punctured wall extending to the epidermis and user distal end 30 of sheet 18 is inserted into the tubular tissue structure 78. Sheet 18 has at least one tether 90 for positioning user distal end 30 within tissue. Alternatively or in combination, a cover such as the dilator cover described in U.S. patent application Ser. No. 11/546,079 titled Dilator, which is expressly incorporated herein by reference, may be used.

As shown in the embodiment of the disclosure depicted in FIG. 3, catheter 10 with sheet 18 is inserted through the skin, the underlying muscle tissue, and through the blood vessel wall (FIG. 3). As shown in FIG. 3, user proximal end 32 of sheet 18 remains outside of the blood vessel wall and at least to the surface of the skin or epidermis. If sheet 18 extends past the surface of the skin, sheet 18 can be trimmed to be flush with the skin if desired. Alternatively, an amount of sheet 18 that extends beyond the skin may be left thereon to allow for clamping or other uses. User distal end 30 of sheet 18 enters the blood vessel when the catheter 10 is inserted into the blood vessel. As discussed above, the submucosal tissue or another extracellular matrix-derived tissue or synthetic bioabsorbable material begins the remodeling process upon insertion of the catheter 10 and sheet 18 into tissue.

Sheath 16 is inserted until the sheet 18 contacts the outside of the vessel where resistance is encountered via tactile stop of cuff 122 or otherwise. Sheet 18 is then be released from sheath 16 by removing retaining wire 94. In such embodiments, catheter 10 is of the type that remains within tissue for an extended period of time for repeated dialysis treatments over time. During such time, sheet 18 remodels and creates a tube of living tissue around catheter 10, thereby sealing the surrounding tissue 124, that was exposed by the incision for catheter 10 placement, from the pathway 126 of catheter 10. Sheet 18 is then held in place via the adherence of the remodeling during any subsequent treatment in which catheter 10 is removed. Pressure from the surrounding tissue causes pathway 126 to collapse in the absence of catheter 10. Additionally, the portion of remodeled sheet 18 within the vessel will likewise collapse in response to force applied by passing blood.

If additional treatment is not expected to be needed for an extended period of time, the exteriorly exposed access point may be stitched or otherwise closed in a fixed manner. In such occasions, removal of the stitches or a shallow incision at the site allows further access to the lumen of sheet 18.

For additional treatment, a replacement catheter 10, without sheet 18 thereon, is then placed within pathway 126 without having to go through the initial placement steps. Rather, replacement catheter expands collapsed pathway 126 as force is axially applied to catheter 10 until distal end 12 of catheter 10 is disposed within the vessel. Accordingly, by creating pathway 126 having remodeled sheet 18 as the interior surface thereof, raw tissue along the pathway is not exposed. Furthermore, such lack of exposed tissue provides a decrease in likelihood of infection forming thereon.

In addition to the tethering of sheet 18 to sheath 16, other means of attachment are envisioned. Such attachment methods include: providing a snap fit or resistance fit, chemically bonding or gluing, and providing a common dilator cover. When attaching sheet 18, or other sealing member, to sheath 16, or a positioning tube, the attachment is provided to allow proper placement of sheet 18 by moving sheath 16 or a positioning tube, and to then allow sheath 16 or the positioning tube to disengage from sheet 18 to leave sheet 18 at the access site when desired. Accordingly, any attachment that achieves these goals is suitable. Embodiments utilizing a snap fit or resistance fit provide for disengagement of sheet 18 when a resistance is encountered that overcomes the snap/resistance attachment of sheet 18. The resistance provided by tubular tissue structure 78 is greater than the resistance provided by general tissue. Accordingly, the holding force of the snap fit/resistance fit is engineered to be greater than the resistance of general tissue, but less than the resistance provided by tubular tissue structure 78. When sheet 18, or cuff 122, encounters tubular tissue structure 78 and sheath 16 or the positioning tube is further urged into tubular tissue structure 78, the snap fit/resistance is overcome to un-bind sheet 18 from the sheath 16 or the positioning tube. Alternatively, the resistance fit is designed to release or be of less strength than the connection between sheet 18 and the surrounding tissue once sheet 18 has significantly remodeled and adhered to the surrounding tissue.

Embodiments using chemical bonding or gluing include chemicals or glues that either dissolve or disengage during the procedure. Such dissolution or disengagement may be a reaction, delayed or immediate, to exposure to solvents within the body, a reaction to air, a reaction to an introduced reagent, or a reaction to an other reagent. Also, the chemical bonding or gluing may be overcome by resistance provided by sheet 18 or cuff 122 encountering the tubular tissue structure 78. Again, the gluing may release or be of less strength than the connection between sheet 18 and the surrounding tissue once sheet 18 has significantly remodeled and adhered to the surrounding tissue.

While certain embodiments of the present disclosure have been described in detail, those familiar with the art to which this disclosure relates will recognize various alternative designs and embodiments for practicing the disclosure as defined by the following claims.

What is claimed is:

1. A device for maintaining access to a bodily cavity comprising:
an elongated element having a tissue wall contact exterior portion and having a length adapted to be inserted into a puncture site so that the length forms intracavity, intermediate, and extracorporeal portions, and a bioabsorbable member releasably attached to the tissue wall contact exterior portion of the elongated element, the bioabsorbable member having intracavity, intermediate, and extracorporeal portions.

2. The device of claim 1, having a pre-insertion configuration, wherein the bioabsorbable member is attached to the elongated element, and having a post-insertion configuration, wherein the bioabsorbable member is unattached from the elongated element.

3. The device of claim 1, wherein the bioabsorbable member is releasably attached to the tissue wall contact exterior portion via a tether.

4. The device of claim 1, wherein the bioabsorbable member is releasably attached to the tissue wall contact exterior portion via a cover.

5. A device for maintaining access to a bodily cavity comprising:
a bioabsorbable tube sized and shaped to extend from outside the body to within the bodily cavity.

6. The device of claim 5, wherein the bioabsorbable tube is configured to remodel into living tissue.

7. The device of claim 5, wherein the bioabsorbable tube includes a lumen.

8. A device for maintaining an access site to a bodily cavity comprising: an elongated element having a tissue wall contact exterior portion and a bioabsorbable member releasably attached to the tissue wall contact exterior portion of the elongated element, the bioabsorbable member having a length to extend from within the cavity to the skin of a body.

9. The device of claim 8, wherein the elongated element further comprises a lumen interior portion.

10. The device of claim 8, wherein the bioabsorbable member includes a lumen.

11. The device of claim 8, wherein the bioabsorbable member is a substantially annular member that encircles the elongated element.

12. The device of claim 8, wherein the bioabsorbable member is releasably attached to the tissue wall contact exterior portion of the elongated element such that the device may be placed at a puncture site when the bioabsorbable member is coupled to the tissue wall contact exterior portion and then uncoupleable such that removal of the elongated element allows the bioabsorbable member to remain at the puncture site.

13. The device of claim 8, wherein the bioabsorbable member is releasably attached to the tissue wall contact exterior portion of the elongated element such that the bioabsorbable member is coupled to the exterior portion before placement of the device at a puncture site and the bioabsorbable member is uncoupleable from the exterior portion after placement of the device at the puncture site.

14. A method of maintaining an access point in tissue, the method comprising the step of:
providing a bioabsorbable member at the access point such that the bioabsorbable member extends from outside the body to within a bodily cavity.

15. The method of claim 14, wherein the bioabsorbable member includes a lumen.

16. A method of maintaining an access point in tissue, the method comprising the step of:
providing a first elongated element having a bioabsorbable member disposed on the exterior thereof, the first elongated element being configured to be introduced into a body with the bioabsorbable member disposed thereon;
removing the first elongated element; and
placing a second elongated element within the bioabsorbable member.

17. The method of claim 16, further including the step of waiting for the bioabsorbable member to remodel before removing the first elongated element.
